SEQUENCE LISTING (25 sequences)

SEQ ID NO: 1 - protein, 266 aa, Sus scrofa
MLRLLVVASLVLYGHSTQDFPETNARVVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 2 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 3 - protein, 255 aa, Homo sapiens
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 4 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 5 - protein, 16 aa, Sus scrofa
MLRLLVVASLVLYGHS

SEQ ID NO: 6 - protein, 14 aa, Sus scrofa
TQDFPETNARVVGG

SEQ ID NO: 7 - protein, 240 aa, Sus scrofa
VVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 8 - protein, 8 aa, Artificial Sequence
  Synthesized: optimized cleavage site
GAAGLGGA

SEQ ID NO: 9 - protein, 8 aa, Artificial Sequence
  Synthesized: optimized cleavage site
GAAGVVGG

SEQ ID NO: 10 - protein, 8 aa, Artificial Sequence
  Synthesized: optimized cleavage site
GAAGLVGG

SEQ ID NO: 11 - protein, 8 aa, Artificial Sequence
  Synthesized: optimized cleavage site
LLVLVVLG

SEQ ID NO: 12 - protein, 8 aa, Artificial Sequence
  Synthesized: optimized cleavage site
LLVLVVGG

SEQ ID NO: 13 - protein, 8 aa, Artificial Sequence
  Synthesized: optimized cleavage site
ASEIVGGR

SEQ ID NO: 14 - protein, 8 aa, Artificial Sequence
  Synthesized: optimized cleavage site
ALLGAAGG

SEQ ID NO: 15 - protein, 8 aa, Artificial Sequence
  Synthesized: optimized cleavage site
ALLGVVGG

SEQ ID NO: 16 - protein, 8 aa, Artificial Sequence
  Synthesized: optimized cleavage site
ALLGAVGG

SEQ ID NO: 17 - protein, 266 aa, Artificial Sequence
  Synthesized: Modified PPE proproteins with heterologous protease cleavage site
MLRLLVVASLVLYGHSTQDFPEGAAGLGGATEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 18 - protein, 266 aa, Artificial Sequence
  Synthesized: Modified PPE proproteins with heterologous protease cleavage site
MLRLLVVASLVLYGHSTQDFPEGAAGVVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 19 - protein, 266 aa, Artificial Sequence
  Synthesized: Modified PPE proproteins with heterologous protease cleavage site
MLRLLVVASLVLYGHSTQDFPEGAAGLVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 20 - protein, 266 aa, Artificial Sequence
  Synthesized: Modified PPE proproteins with heterologous protease cleavage site
MLRLLVVASLVLYGHSTQDFPELLVLVVLGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 21 - protein, 266 aa, Artificial Sequence
  Synthesized: Modified PPE proproteins with heterologous protease cleavage site
MLRLLVVASLVLYGHSTQDFPELLVLVVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 22 - protein, 266 aa, Artificial Sequence
  Synthesized: Modified PPE proproteins with heterologous protease cleavage site
MLRLLVVASLVLYGHSTQDFPEASEIVGGRTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 23 - protein, 266 aa, Artificial Sequence
  Synthesized: Modified PPE proproteins with heterologous protease cleavage site
MLRLLVVASLVLYGHSTQDFPEALLGAAGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 24 - protein, 266 aa, Artificial Sequence
  Synthesized: Modified PPE proproteins with heterologous protease cleavage site
MLRLLVVASLVLYGHSTQDFPEALLGVVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 25 - protein, 266 aa, Artificial Sequence
  Synthesized: Modified PPE proproteins with heterologous protease cleavage site
MLRLLVVASLVLYGHSTQDFPEALLGAVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

(19) United States
(12) Patent Application Publication
(10) Pub. No.: US 2023/0348881 A1
(43) Pub. Date: Nov. 2, 2023

(54) MODIFIED SERINE PROTEASE PROPROTEINS

(71) Applicants: Onchilles Pharma, Inc., San Diego, CA (US); The University of Chicago, Chicago, IL (US)

(72) Inventors: Lev BECKER, San Diego, CA (US); Marc NASOFF, Rancho Santa Fe, CA (US)

(21) Appl. No.: 18/022,004

(22) PCT Filed: Aug. 18, 2021
(86) PCT No.: PCT/US2021/046467

§ 371 Date: Feb. 17, 2023

Related U.S. Application Data
(60) Provisional application No. 63/067,059, filed on Aug. 18, 2020.

(51) Int. Cl. A61P 35/00; C12N 9/64

(52) U.S. Cl. CPC C12N 9/6448; A61P 35/00; C12Y 304/21036

(57) ABSTRACT

Provided are modified serine protease proproteins, such as porcine pancreatic elastase (PPE) proproteins, comprising a heterologous protease cleavage site that is cleavable by a tumor site protease, and related pharmaceutical compositions and methods of use for treating diseases such as cancers.

### CROSS-REFERENCE TO RELATED APPLICATIONS

This application claims the benefit under 35 U.S.C. § 119(e) to U.S. Application No. 63/067,059, filed Aug. 18, 2020, which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is OPNI_002_01WO_ST25.txt. The text file is about 35 KB, was created on Aug. 16, 2021, and is being submitted electronically via EFS-Web.

### BACKGROUND

### Technical Field

The present disclosure relates to modified serine protease proproteins, such as porcine pancreatic elastase (PPE) proproteins, comprising a heterologous protease cleavage site that is cleavable by a tumor site protease, and related pharmaceutical compositions and methods of use for treating diseases such as cancers.

### Description of the Related Art

Precision medicine, which is designed to optimize efficiency or therapeutic benefit for particular groups of patients by using genetic or molecular profiling, has gained tremendous traction for treating cancer. Identifying the specific genomic abnormalities that (i) confer risk of developing cancer, (ii) influence tumor growth, and (iii) regulate metastasis have defined how cancer is diagnosed, determined how targeted therapies are developed and implemented, and shaped cancer prevention strategies.

The need for precision medicine in cancer is largely based on the failure to identify targetable properties in tumor cells that distinguish them from healthy, non-cancer cells. Indeed, although radiation and/or chemotherapies have the capacity to effectively kill many if not most cancer cells, their efficacy is severely limited by cytotoxic effects on non-cancer cells. These findings demonstrate that rapid cell division, a property targeted by radiation therapy and chemotherapy, is not unique enough to cancer cells to achieve the specificity required to limit extensive side effects.

It has been shown that certain serine protease enzymes are selectively toxic to cancer cells but relatively non-toxic to normal or otherwise healthy cells. However, there is a need in the art to identify optimal enzymes that are capable of such selective cancer cell-toxicity, and refine the clinical utility of such enzymes.

### BRIEF SUMMARY

Embodiments of the present disclosure include a modified serine protease proprotein, comprising in an N-terminal to C-terminal orientation, a signal peptide, a modified activation peptide, and a peptidase domain, wherein the modified activation peptide comprises a heterologous protease cleavage site that is cleavable by a protease selected from a metalloprotease, an aspartyl protease, and a cysteine protease. In some embodiments, the serine protease is selected from porcine pancreatic elastase (PPE), human neutrophil elastase (ELANE), human cathepsin G (CTSG), and human proteinase 3 (PR3).

In some embodiments, the modified serine protease proprotein comprises, consists, or consists essentially of an amino acid sequence that is at least 80, 85, 90, 95, 98, or 100% identical to a sequence selected from Table S1, and which comprises or retains the heterologous protease cleavage site.

In some embodiments, the metalloprotease, aspartyl protease, or cysteine protease is selected from matrix metalloproteinase-12 (MMP12), cathepsin D (CTSD), cathepsin C (CTSD), and cathepsin L (CTSL). In specific embodiments, the heterologous protease cleavage site is selected from Table S3, for example, the MMP12 cleavage site of SEQ ID NO: 8, the CTSD cleavage site of SEQ ID NO: 11, the CTSC cleavage site of SEQ ID NO: 13, or the CTSL cleavage site of SEQ ID NO: 14.

In some embodiments, the modified serine protease proprotein does not substantially bind to a serine protease inhibitor (Serpin) in vitro or in vivo, optionally wherein the Serpin includes alpha-1 antitrypsin (A1AT). In some embodiments, the modified serine protease proprotein is substantially inactive as a serine protease in its proprotein form. In some embodiments, protease cleavage of the heterologous protease cleavage site, for example, at a cancer or tumor site in vivo, generates an active peptidase domain (or active serine protease domain), which has increased serine protease activity relative to the proprotein. In some embodiments, the serine protease activity of the active peptidase domain is increased by about or at least about 2-fold, 5-fold, 10-fold, 50-fold, 100-fold, 500-fold, or 1000-fold or more relative to that of the proprotein.

In some embodiments, protease cleavage of the heterologous protease cleavage site, for instance, at a cancer or tumor site in vivo, generates an active peptidase domain (or active serine protease domain), which has increased cancer cell-killing activity relative to the proprotein. In some embodiments, the cancer cell-killing activity of the active peptidase domain is increased by about or at least about 2-fold, 5-fold, 10-fold, 50-fold, 100-fold, 500-fold, or 1000-fold or more relative to that of the proprotein.

In some embodiments:
the serine protease is PPE, and the active peptidase domain comprises, consists, or consists essentially of an amino acid sequence that is at least 80, 85, 90, 95, 98, or 100% identical to residues 31-266 of SEQ ID NO: 1;
the serine protease is human ELANE, and the active peptidase domain comprises, consists, or consists essentially of an amino acid sequence that is at least 80, 85, 90, 95, 98, or 100% identical to residues 30-247 of SEQ ID NO: 2;
the serine protease is human CTSG, and the active peptidase domain comprises, consists, or consists essentially of an amino acid sequence that is at least 80, 85, 90, 95, 98, or 100% identical to residues 21-243 of SEQ ID NO: 3; or
the serine protease is human PR3, and the active peptidase domain comprises, consists, or consists essentially of an amino acid sequence that is at least 80, 85, 90, 95, 98, or 100% identical to residues 28-248 of SEQ ID NO: 4.

Certain embodiments include a modified porcine pancreatic elastase (PPE) proprotein, comprising in an N-terminal to C-terminal orientation, a signal peptide, a modified activation peptide relative to SEQ ID NO: 6 (wild-type PPE activation peptide), and a PPE peptidase domain, wherein the modified activation peptide is not substantially cleavable by trypsin and comprises a heterologous protease cleavage site that is cleavable by a protease selected from a metalloprotease, an aspartyl protease, and a cysteine protease.

In some embodiments, the protease is selected from matrix metalloproteinase-12 (MMP12), cathepsin D (CTSD), cathepsin C (CTSC), and cathepsin L (CTSL). In some embodiments, the heterologous protease cleavage site comprises, consists, or consists essentially of an amino acid sequence selected from Table S3.

In some embodiments:
the heterologous protease cleavage site is selected from SEQ ID NOs: 8-10, and is cleavable by MMP12;
the heterologous protease cleavage site is selected from SEQ ID NOs: 11-12, and is cleavable by CTSD;
the heterologous protease cleavage site is SEQ ID NO: 13, and is cleavable by CTSC; or
the heterologous protease cleavage site is selected from SEQ ID NOs: 14-16, and is cleavable by CTSL.

In some embodiments, the signal peptide comprises the amino acid sequence set forth in SEQ ID NO: 5, or a variant thereof, and wherein the PPE peptidase domain comprises the amino acid sequence set forth in SEQ ID NO: 7, or an amino acid sequence that is at least 80, 85, 90, 95, 98, or 99% identical to SEQ ID NO: 7. In some embodiments, the modified PPE proprotein comprises, consists, or consists essentially of an amino acid sequence that is at least 80, 85, 90, 95, 98, or 100% identical to a sequence selected from Table S4, and which retains the heterologous protease cleavage site.

In some embodiments, the modified PPE proprotein does not substantially bind to a serine protease inhibitor (Serpin) in vitro or in vivo, optionally wherein the Serpin includes alpha-1 antitrypsin (A1AT). In some embodiments, the modified PPE proprotein is substantially inactive as a serine protease in its PPE proprotein form.

In some embodiments, protease cleavage of the heterologous protease cleavage site, for instance, at a cancer or tumor site in vivo, generates an active PPE peptidase domain (or active PPE protein), which has increased serine protease activity relative to the PPE proprotein. In some embodiments, the serine protease activity of the active PPE protein is increased by about or at least about 2-fold, 5-fold, 10-fold, 50-fold, 100-fold, 500-fold, or 1000-fold or more relative to that of the PPE proprotein.

In some embodiments, protease cleavage of the heterologous protease cleavage site, for example, at a cancer or tumor site in vivo, generates an active PPE peptidase domain (or active PPE protein), which has increased cancer cell-killing activity relative to the PPE proprotein. In some embodiments, the cancer cell-killing activity of the active PPE protein is increased by about or at least about 2-fold, 5-fold, 10-fold, 50-fold, 100-fold, 500-fold, or 1000-fold or more relative to that of the PPE proprotein.

Also included are recombinant nucleic acid molecules encoding a modified serine protease proprotein, for example, a modified PPE proprotein, described herein, a vector comprising the recombinant nucleic acid molecule, or a host cell comprising the recombinant nucleic acid molecule or the vector. Certain embodiments include methods of producing a modified serine protease proprotein, for example, a modified PPE proprotein, as described herein, comprising culturing the host cell of claim under culture conditions suitable for the expression of the proprotein, and isolating the proprotein from the culture.

Some embodiments include pharmaceutical compositions, comprising a modified serine protease proprotein, for instance, a modified PPE proprotein, as described herein, or an expressible polynucleotide encoding the proprotein, and a pharmaceutically acceptable carrier.

Certain embodiments include methods of treating, ameliorating the symptoms of, and/or reducing the progression of, a cancer in a subject in need thereof, comprising administering to the subject pharmaceutical composition described herein. In some embodiments, the cancer is a primary cancer or a metastatic cancer, and is selected from one or more of melanoma (optionally metastatic melanoma), breast cancer (optionally triple-negative breast cancer, TNBC), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In some embodiments, the modified serine protease proprotein, for example, the modified PPE proprotein, is activated by protease cleavage of the heterologous protease cleavage site in a cell or tissue, such as a cancer or tumor cell or tissue, to generate an active peptidase domain, for example, an active PPE peptidase domain, wherein the active peptidase domain has increased serine protease activity and/or cancer cell-killing activity relative to the proprotein. In some embodiments, the active peptidase domain increases cancer cell-killing in the subject by about or at least about 2-fold, 5-fold, 10-fold, 50-fold, 100-fold, 500-fold, or 1000-fold or more relative to a control or reference. In some embodiments, the active peptidase domain results in tumor regression in the subject, optionally as indicated by a statistically significant decrease in the amount of viable tumor or tumor mass, optionally at least about a 10%, 20%, 30%, 40%, 50% or more decrease in tumor mass.

Some embodiments comprise administering the pharmaceutical composition to the subject by parenteral administration. In some embodiments, the parenteral administration is intravenous administration.

### BRIEF DESCRIPTION OF THE FIGURES

FIG. 1 shows that activated PPE proteins kill cancer cells but are non-toxic to normal or non-cancer cells. Shown is the treatment of human cancer cells (MDA-MB-231, a triple-negative breast cancer (TNBC) cell line; MEL888, a melanoma cell line; and A549, a lung adenocarcinoma cell line) and non-cancer cells (HMDMs, or human monocyte-derived macrophages isolated from healthy donors) with serum-free media (SFM), activated native PPE (PPE), activated recombinant PPE (rPPE), and trypsin.

FIG. 2 shows that the PPE proprotein (pro-rPPE) does not bind to A1AT. The results show full recovery of protease activity for pro-rPPE following isolation from the A1AT solution, suggesting that pro-rPPE does not bind A1AT. In contrast, protease activity of activated PPE (rPPE) was attenuated by A1AT when subjected to an identical procedure. Inset: immunoblotting for A1AT pre-and post-purification of pro-rPPE.

FIGS. 3A-3B show that the MMP12 protease cleaved the exemplary modified PPE proteins designated as Mutant 2 (3A) and Mutant 3 (3B). FIG. 3A also shows that trypsin cleaved the wild-type PPE as a control.

FIG. 4A shows that exemplary modified PPE proteins were catalytically-active after incubation by MMP12. FIG. 4B shows that exemplary modified PPE proproteins were catalytically-active after incubation with MMP12, partially-active after incubation with MMP7, and catalytically-inactive after incubation with trypsin or no protease. In contrast, the wild-type PPE proprotein was catalytically-active after incubation with trypsin but catalytically-inactive after incubation with MMP12 or no protease.

FIG. 5 shows that exemplary modified PPE proteins demonstrated significant cancer cell-killing activity after incubation with (and activation by) the MMP12 protease.

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer’s specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

The term “about” is used to indicate that a value includes the inherent variation of error for the measurement or quantitation method, for example, a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

An “antagonist” refers to a biological or chemical agent that interferes with or otherwise reduces the physiological action of another agent or molecule. In some instances, the antagonist specifically binds to the other agent or molecule. Included are full and partial antagonists.

An “agonist” refers to a biological or chemical agent that increases or enhances the physiological action of another agent or molecule. In some instances, the agonist specifically binds to the other agent or molecule. Included are full and partial agonists.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligosaccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “half maximal effective concentration” or “EC₅₀” refers to the concentration of an agent (for example, a modified serine protease proprotein, or an active/activated peptidase domain thereof) as described herein at which it induces a response halfway between the baseline and maximum after some specified exposure time; the EC₅₀ of a graded dose response curve therefore represents the concentration of a compound at which 50% of its maximal effect is observed. EC50 also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC50” and the Hill slope, or it can be determined from the data directly, using routine knowledge in the art. In some embodiments, the EC₅₀ of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅0 value of about 1 nM or less.

The “half-life” of an agent can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The term “heterologous” refers to a feature or element (e.g., protease cleavage site) in a polypeptide or encoding polynucleotide that is derived from a different source than the wild-type polypeptide or encoding polynucleotide, for example, a feature from a different species than the wild-type, or a non-natural, engineered feature.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, or 1000-fold more than the amount produced by no composition (e.g., the absence of agent) or a control composition. A “decreased” or “reduced” amount is typically a “statistically significant” amount, and may include a decrease that about or at least about 1.1, 1.2, 1.5, 2, 3, 4, 5, 6, 7, 8, 9, 10, 15, 20, 30, 40, 50, 60, 70, 80, 90, 100, 200, 300, 400, 500, or 1000-fold less than the amount produced by no composition (e.g., the absence of an agent) or a control composition. Examples of comparisons and “statistically significant” amounts are described herein.

The terms “polypeptide,” “protein”, and “peptide” are used interchangeably and refer to a polymer of amino acids not limited to any particular length. The term “enzyme” includes polypeptide or protein catalysts. As used herein a “proprotein”, “proenzyme”, or “zymogen” refers to an inactive (or substantially inactive) protein or enzyme, which typically is activated by protease cleavage of an activation peptide to generate an active protein or enzyme. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotide, regardless of the length of the coding sequence itself, may be combined with other sequences, for example, expression control sequences.

“Expression control sequences” include regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, which have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Eukaryotic promoters can often, but not always, contain “TATA” boxes and “CAT” boxes. Prokaryotic promoters contain Shine- Dalgarno sequences in addition to the -10 and -35 consensus sequences.

A large number of promoters, including constitutive, inducible and repressible promoters, from a variety of different sources are well known in the art. Representative sources include for example, viral, mammalian, insect, plant, yeast, and bacterial cell types), and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bidirectional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include the Tet system, (U.S. Pats. 5,464,758 and 5,814,618), the Ecdysone inducible system (No et al., Proc. Natl. Acad. Sci. (1996) 93 (8): 3346-3351; the T-RExTM system (Invitrogen Carlsbad, CA), LacSwitch® (Stratagene, (San Diego, CA) and the Cre-ERT tamoxifen inducible recombinase system (Indra et al. Nuc. Acid. Res. (1999) 27 (22): 4324-4327; Nuc. Acid. Res. (2000) 28 (23): e99; U.S. Pat. No. 7,112,715; and Kramer & Fussenegger, Methods Mol. Biol. (2005) 308: 123-144) or any promoter known in the art suitable for expression in the desired cells.

An “expressible polynucleotide” includes a cDNA, RNA, mRNA or other polynucleotide that comprises at least one coding sequence and optionally at least one expression control sequence, for example, a transcriptional and/or translational regulatory element, and which can express an encoded polypeptide (for example, a modified serine protease proprotein, such as modified PPE proprotein) upon introduction into a cell, for example, a cell in a subject.

In some embodiments, the expressible polynucleotide is a modified RNA or modified mRNA polynucleotide, for example, a non-naturally occurring RNA analog. In certain embodiments, the modified RNA or mRNA polypeptide comprises one or more modified or non-natural bases, for example, a nucleotide base other than adenine (A), guanine (G), cytosine (C), thymine (T), and/or uracil (U). In some embodiments, the modified mRNA comprises one or more modified or non-natural internucleotide linkages. Expressible RNA polynucleotides for delivering an encoded therapeutic polypeptide are described, for example, in Kormann et al., Nat Biotechnol. 29:154-7, 2011; and U.S. Application Nos. 2015/0111248; 2014/0243399; 2014/0147454; and 2013/0245104, which are incorporated by reference in their entireties.

In some embodiments, various viral vectors that can be utilized to deliver an expressible polynucleotide include adenoviral vectors, herpes virus vectors, vaccinia virus vectors, adeno-associated virus (AAV) vectors, and retroviral vectors. In some instances, the retroviral vector is a derivative of a murine or avian retrovirus, or is a lentiviral vector. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of additional retroviral vectors can incorporate multiple genes. All of these vectors can transfer or incorporate a gene for a selectable marker so that transduced cells can be identified and generated. By inserting a polypeptide sequence of interest into the viral vector, along with another gene that encodes the ligand for a receptor on a specific target cell, for example, the vector may be made target specific. Retroviral vectors can be made target specific by inserting, for example, a polynucleotide encoding a protein. Illustrative targeting may be accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector.

In certain instances, the expressible polynucleotides described herein are engineered for localization within a cell, potentially within a specific compartment such as the nucleus, or are engineered for secretion from the cell or translocation to the plasma membrane of the cell. In exemplary embodiments, the expressible polynucleotides are engineered for nuclear localization.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the proteins/polypeptides described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60,70, 80, 90, 100, 110, 120, 130, 140, 150 or more amino acids or nucleotides and which substantially retain at least one activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent described herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

### Modified Serine Protease Proproteins

Embodiments of the present disclosure relate to modified serine protease proproteins, comprising a heterologous protease cleavage site that is cleavable by an alternate protease, for example, a protease that is found at relatively higher levels in a cancer tissue or at a tumor site. Serine proteases are a class of enzymes that cleave peptide bonds in proteins, in which serine serves as the nucleophilic amino acid at the enzyme’s active site. Generally, serine proteases are produced as an inactive “proprotein” (or “proenzyme”, “zymogen”) composed of a signal peptide, an activation peptide comprising a native or wild-type protease cleavage site, and an active peptidase domain. The proprotein is activated by protease cleavage of the activation peptide to release the enzymatically-active, peptidase domain.

As described herein, certain serine proteases are able to kill cancer cells upon direct contact with or administration to tumors (e.g., intra-tumoral administration), irrespective of their genetic abnormalities, and are relatively harmless to non-cancerous or healthy cells (see, for example, Example 1; WO 2018/232273 and WO/2020/132465). However, one barrier to anti-tumor efficacy of such serine proteases in vivo is that parenteral administration achieves relatively low levels of the mature or active peptidase domain in cancer tissues or at tumor sites. Here, high blood or serum levels of serine protease inhibitors (Serpins) inhibit or otherwise impair the catalytic activity and cancer cell-killing capabilities of the mature or active peptidase domain. For instance, blood or serum levels of alpha-1 antitrypsin (A1AT; UniProtKB - P01009) are about 300 mg/dL.

But Serpins such as A1AT do not bind to or inhibit full-length serine protease proproteins, only the mature or active peptidase domains. Thus, systemic delivery as a “proprotein” (or “proenzyme”, “zymogen”) protects the active peptidase domain from binding to, and being inactivated by, Serpins in blood. However, wild-type serine protease proproteins are activated by specific circulating proteases, most of which are present at very low levels in cancer tissues or tumors. As one example, wild-type porcine pancreatic elastase (PPE) proproteins are activated by trypsin, which is absent or present at only at very low levels in cancer tissues or tumors. A wild-type serine protease proprotein would not be selectively activated in cancer tissues or tumor sites following system administration, but would instead be activated systemically, for example, in blood, where Serpins would bind to and impair its catalytic activity and cancer cell-killing capabilities.

As noted above, embodiments of the present disclosure therefore relate to modified serine protease proproteins, in which the activation peptide containing the native protease cleavage site is replaced or otherwise modified so that it is not cleavable (or not substantially cleavable) by its native protease under suitable conditions (e.g., in vivo, in vitro, for example, using a colorimetric substrate activity assay, see the Examples), but is instead cleavable by a protease that is present at relatively higher levels in cancer tissues or at tumor sites. Certain embodiments include a modified serine protease proprotein, comprising in an N-terminal to C-terminal orientation, a signal peptide, a modified activation peptide, and a peptidase domain, wherein the modified activation peptide comprises a heterologous protease cleavage site that is cleavable by a tumor site protease, for example, a metalloprotease, an aspartyl protease, or a cysteine protease. In particular embodiments, the heterologous protease cleavage site is cleavable by matrix metalloproteinase-12 (MMP12), cathepsin D (CTSD), cathepsin C (CTSC), or cathepsin L (CTSL).

Examples of serine proteases that can be modified in this way include porcine pancreatic elastase (PPE), human neutrophil elastase (ELANE), human cathepsin G (CTSG), and human proteinase 3 (PR3). The amino acid sequences of exemplary wild-type serine proteases are provided in Table S1 below.

TABLE S1

Wild-Type Serine Protease Sequences

| | | |
|---|---|---|
| Name | Sequence | SEQ ID NO: |
| WT PPE proprotein, peptidase domain residues 31-266, signal/activation peptide domains underlined | MLRLLVVASLVLYGHSTQDFPETNARVVGGTEAQRNSWPSQ ISLQYRSGSSWAHTCGGTLIRQNWVMTAAHCVDRELTFRVV VGEHNLNQNDGTEQYVGVQKIVVHPYWNTDDVAAGYDIALL RLAQSVTLNSYVQLGVLPRAGTILANNSPCYITGWGLTRTN GQLAQTLQQAYLPTVDYAICSSSSYWGSTVKNSMVCAGGDG VRSGCQGDSGGPLHCLVNGQYAVHGVTSFVSRLGCNVTRKP TVFTRVSAYISWINNVIASN | 1 |
| WT human ELANE, peptidase domain residues 30-247, or residues 30-267, signal/activation peptide domains underlined | MTLGRRLACLFLACVLPALLLGGTALASEIVGGRRARPHAW PFMVSLQLRGGHFCGATLIAPNFVMSAAHCVANVNVRAVRV VLGAHNLSRREPTRQVFAVQRIFENGYDPVNLLNDIVILQL NGSATINANVQVAQLPAQGRRLGNGVQCLAMGWGLLGRNRG IASVLQELNVTVVTSLCRRSNVCTLVRGRQAGVCFGDSGSP LVCNGLIHGIASFVRGGCASGLYPDAFAPVAQFVNWIDSII QRSEDNPCPHPRDPDPASRTH | 2 |
| WT human CTSG, peptidase domain residues 21-243, or residues 21-255, signal/activation peptide domains underlined | MQPLLLLLAFLLPTGAEAGEIIGGRESRPHSRPYMAYLQIQ SPAGQSRCGGFLVREDFVLTAAHCWGSNINVTLGAHNIQRR ENTQQHITARRAIRHPQYNQRTIQNDIMLLQLSRRVRRNRN VNPVALPRAQEGLRPGTLCTVAGWGRVSMRRGTDTLREVQL RVQRDRQCLRIFGSYDPRRQICVGDRRERKAAFKGDSGGPL LCNNVAHGIVSYGKSSGVPPEVFTRVSSFLPWIRTTMRSFK LLDQMETPL | 3 |
| WT Human PR3, peptidase domain residues 28-248, | MAHRPPSPALASVLLALLLSGAARAAEIVGGHEAQPHSRPY MASLQMRGNPGSHFCGGTLIHPSFVLTAAHCLRDIPQRLVN VVLGAHNVRTQEPTQQHFSVAQVFLNNYDAENKLNDVLLIQ LSSPANLSASVATVQLPQQDQPVPHGTQCLAMGWGRVGAHD | 4 |
| signal/activation peptide domains underlined | PPAQVLQELNVTVVTFFCRPHNICTFVPRRKAGICFGDSGG PLICDGIIQGIDSFVIWGCATRLFPDFFTRVALYVDWIRST LRRVEAKGRP | |

Thus, in certain embodiments, a modified serine protease comprises, consists, or consists essentially of an amino acid sequence that is at least 80, 85, 90, 95, 98, or 100% identical (or any range or value derivable therein) to a sequence selected from Table S1, and comprises a heterologous protease cleavage site that is cleavable by a protease selected from a metalloprotease, an aspartyl protease, and a cysteine protease. In specific embodiments, the heterologous protease cleavage site is cleavable by MMP12, CTSD, CTSC, or CTSL. Exemplary heterologous protease cleavage sites are provided in Table S3, including the MMP12 cleavage site of SEQ ID NO: 8, the CTSD cleavage site of SEQ ID NO: 11, the CTSC cleavage site of SEQ ID NO: 13, and the CTSL cleavage site of SEQ ID NO: 14.

In some embodiments, the modified serine protease proprotein does not substantially bind to a serine protease inhibitor (Serpin) in vitro or in vivo, for example, wherein the Serpin is alpha-1 antitrypsin (A1AT). In some embodiments, the modified serine protease proprotein is substantially inactive as a serine protease in its proprotein form.

In particular embodiments, protease cleavage of the heterologous protease cleavage site, for instance, at a cancer or tumor site in vivo, generates an active peptidase domain (or active serine protease domain), which has increased serine protease activity relative to the proprotein. In particular embodiments, the serine protease activity of the active peptidase domain is increased by about or at least about 2-fold, 5-fold, 10-fold, 50-fold, 100-fold, 500-fold, or 1000-fold or more relative to that of the proprotein. In some embodiments, protease cleavage of the heterologous protease cleavage site, optionally at a cancer or tumor site in vivo, generates an active peptidase domain (or active serine protease domain), which has increased cancer cell-killing activity relative to the proprotein. In some embodiments, the cancer cell-killing activity of the active peptidase domain is increased by about or at least about 2-fold, 5-fold, 10-fold, 50-fold, 100-fold, 500-fold, or 1000-fold or more relative to that of the proprotein.

In particular embodiments, the serine protease is PPE, and the active peptidase domain comprises, consists, or consists essentially of an amino acid sequence that is at least 80, 85, 90, 95, 98, or 100% identical (or any range or value derivable therein) to SEQ ID NO: 7, or residues 31-266 of SEQ ID NO: 1. In some embodiments, the serine protease is human ELANE, and the active peptidase domain comprises, consists, or consists essentially of an amino acid sequence that is at least 80, 85, 90, 95, 98, or 100% identical (or any range or value derivable therein) to residues 30-247 of SEQ ID NO: 2. In some embodiments, the serine protease is human CTSG, and the active peptidase domain comprises, consists, or consists essentially of an amino acid sequence that is at least 80, 85, 90, 95, 98, or 100% identical (or any range or value derivable therein) to residues 21-243 of SEQ ID NO: 3. In some embodiments, the serine protease is human PR3, and the active peptidase domain comprises, consists, or consists essentially of an amino acid sequence that is at least 80, 85, 90, 95, 98, or 100% identical (or any range or value derivable therein) to residues 28-248 of SEQ ID NO: 4.

Specific embodiments relate to modified porcine pancreatic elastase (PPE) proproteins, comprising a modified activation peptide relative to the wild-type activation peptide of SEQ ID NO: 6, which is not cleavable by trypsin but is rather cleavable by an alternate protease, for example, a protease that is found at relatively higher levels in a cancer tissue or at a tumor site. As above, PPE is produced as an inactive proprotein (or proenzyme, zymogen) composed of a signal peptide, an activation peptide, and a PPE peptidase domain. The wild-type PPE proprotein is activated by trypsin cleavage of the activation peptide to release the enzymatically-active PPE peptidase domain, or PPE protein. The amino acid sequences of wild-type PPE and its domains are provided in Table S2.

TABLE S2

Wild-Type PPE Sequences

| Name | Sequence | SEQ ID NO: |
|---|---|---|
| WT PPE proprotein, activation peptide underlined | MLRLLVVASLVLYGHSTQDFPETNARVVGGTEAQRNSWPSQIS LQYRSGSSWAHTCGGTLIRQNWVMTAAHCVDRELTFRVVVGEH NLNQNDGTEQYVGVQKIVVHPYWNTDDVAAGYDIALLRLAQSV TLNSYVQLGVLPRAGTILANNSPCYITGWGLTRTNGQLAQTLQ QAYLPTVDYAICSSSSYWGSTVKNSMVCAGGDGVRSGCQGDSG GPLHCLVNGQYAVHGVTSFVSRLGCNVTRKPTVFTRVSAYISW INNVIASN | 1 |
| WT PPE signal peptide | MLRLLVVASLVLYGHS | 5 |
| WT PPE activation peptide; trypsin cleavage site underlined | TQDFPETNAR/VVGG | 6 |
| WT PPE protein; peptidase domain | VVGGTEAQRNSWPSQISLQYRSGSSWAHTCGGTLIRQNWVMTA AHCVDRELTFRVVVGEHNLNQNDGTEQYVGVQKIVVHPYWNTD DVAAGYDIALLRLAQSVTLNSYVQLGVLPRAGTILANNSPCYI TGWGLTRTNGQLAQTLQQAYLPTVDYAICSSSSYWGSTVKNSM VCAGGDGVRSGCQGDSGGPLHCLVNGQYAVHGVTSFVSRLGCN VTRKPTVFTRVSAYISWINNVIASN | 7 |

Thus, certain embodiments include modified PPE proproteins, in which the activation peptide is replaced or otherwise modified so that it is not cleavable (or not substantially cleavable) by trypsin, but is instead cleavable by a protease that is present at relatively higher levels in cancer tissues or at tumor sites. For example, certain modified activation peptides comprise a heterologous protease cleavage site that is cleavable by a metalloprotease, an aspartyl protease, or a cysteine protease. In particular embodiments, the heterologous protease cleavage site is cleavable by MMP12, CTSD, CTSC, or CTSL. The amino acid sequences of exemplary protease cleavage sites are provided in Table S3.

TABLE S3

Exemplary Protease Cleavage Sites

| Name | Sequence | SEQ ID NO: |
|---|---|---|
| MMP12 cleavage site, cleavage optimized | GAAG/LGGA | 8 |
| MMP12 cleavage site, PPE optimized | GAAG/VVGG | 9 |
| MMP12 cleavage site, | GAAG/LVGG | 10 |
| balanced | | |
| CTSD cleavage site, cleavage optimized | LLVL/VVLG | 11 |
| CTSD cleavage site; PPE optimized | LLVL/VVGG | 12 |
| CTSC cleavage site, cleavage optimized | ASEI/VGGR | 13 |
| CTSL cleavage site, cleavage optimized | ALLG/AAGG | 14 |
| CTSL cleavage site, PPE optimized | ALLG/VVGG | 15 |
| CTSL cleavage site, balanced | ALLG/AVGG | 16 |

Cleavage Site Residues = P1 P2 P3 P4/P1′ P2′ P3′ P4′
Cleavage optimized = mutations in WT PPE protein residues P1′-P4’ that maximize activating enzyme preference
PPE optimized = retains WT PPE protein residues P1′-P4’ after cleavage
Balanced = conservative mutations in PPE protein residues P1′-P4’ to balance activating enzyme preference with retention of WT PPE protein sequence

In certain embodiments, a modified activation peptide has a heterologous protease cleavage site that comprises, consists, or consists essentially of an amino acid sequence selected from Table S3. For example, in some embodiments, the modified activation peptide comprises a protease cleavage site selected from SEQ ID NOs: 8-10, and is cleavable by MMP12; or the modified activation peptide comprises a protease cleavage site selected from SEQ ID NOs: 11-12, and is cleavable by CTSD; or the modified activation peptide comprises a protease cleavage site of SEQ ID NO: 13, and is cleavable by CTSC; or the modified activation peptide comprises a protease cleavage site selected from SEQ ID NOs: 14-16, and is cleavable by CTSL.

Examples of modified PPE proproteins having a heterologous protease cleavage site, as described herein, are provided in Table S4 below.

TABLE S4

Exemplary Modified PPE Proprotein sequences

| | Sequence | SEQ ID NO: |
|---|---|---|
| Mutant 1 | MLRLLVVASLVLYGHSTQDFPEGAAGLGGATEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG DSGGPLHCLVNGQYAVHGVTSFVSRLGCNVTRKPTVFTRVSAYISWINNVIAS N | 17 |
| Mutant 2 | MLRLLVVASLVLYGHSTQDFPEGAAGVVGGTEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG DSGGPLHCLVNGQYAVHGVTSFVSRLGCNVTRKPTVFTRVSAYISWINNVIAS N | 18 |
| Mutant 3 | MLRLLVVASLVLYGHSTQDFPEGAAGLVGGTEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG | 19 |
| | DSGGPLHCLVNGQYAVHGVTSFVSRLGCNVTRKPTVFTRVSAYISWINNVIAS N | |
| Mutant 4 | MLRLLVVASLVLYGHSTQDFPELLVLVVLGTEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG DSGGPLHCLVNGQYAVHGVTSFVSRLGCNVTRKPTVFTRVSAYISWINNVIAS N | 20 |
| Mutant 5 | MLRLLVVASLVLYGHSTQDFPELLVLVVGGTEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG DSGGPLHCLVNGQYAVHGVTSFVSRLGCNVTRKPTVFTRVSAYISWINNVIAS N | 21 |
| Mutant 6 | MLRLLVVASLVLYGHSTQDFPEASEIVGGRTEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG DSGGPLHCLVNGQYAVHGVTSFVSRLGCNVTRKPTVFTRVSAYISWINNVIAS N | 22 |
| Mutant 7 | MLRLLVVASLVLYGHSTQDFPEALLGAAGGTEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG DSGGPLHCLVNGQYAVHGVTSFVSRLGCNVTRKPTVFTRVSAYISWINNVIAS N | 23 |
| Mutant 8 | MLRLLVVASLVLYGHSTQDFPEALLGVVGGTEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG DSGGPLHCLVNGQYAVHGVTSFVSRLGCNVTRKPTVFTRVSAYISWINNVIAS N | 24 |
| Mutant 9 | MLRLLVVASLVLYGHSTQDFPEALLGAVGGTEAQRNSWPSQISLQYRSGSSWA HTCGGTLIRQNWVMTAAHCVDRELTFRVVVGEHNLNQNDGTEQYVGVQKIVVH PYWNTDDVAAGYDIALLRLAQSVTLNSYVQLGVLPRAGTILANNSPCYITGWG LTRTNGQLAQTLQQAYLPTVDYAICSSSSYWGSTVKNSMVCAGGDGVRSGCQG DSGGPLHCLVNGQYAVHGVTSFVSRLGCNVTRKPTVFTRVSAYISWINNVIAS N | 25 |

In some instances, any one or more of the modified PPE proproteins of Table S4 are included in embodiments of the present disclosure. Thus, in certain embodiments, a modified PPE proprotein comprises, consists, or consists essentially of an amino acid sequence selected from Table S4, or an amino acid sequence that is at least 80, 85, 90, 95, 98, 99, or 100% identical (or any range or value derivable therein) to a sequence selected from Table S4, and which retains the heterologous protease cleavage site (underlined). In certain embodiments, the wild-type signal peptide from Table S4 is modified or otherwise replaced with a different signal peptide. In some instances, any one or more of the modified PPE proproteins of Table S4 are excluded from certain embodiments of the disclosure.

In some embodiments, the PPE proprotein comprises the wild-type PPE peptidase domain (SEQ ID NO: 7), and in some embodiments, the PPE peptidase domain is a variant or fragment thereof, for example, which comprises an amino acid sequence that is at least 80, 85, 90, 95, 98, 99, or 100% identical (or any range or value derivable therein) to SEQ ID NO: 7, and which has serine protease activity and/or cancer cell-killing activity. Certain PPE peptidase domain variants (of SEQ ID NO: 7) have increased cancer cell-killing activity, and/or reduced binding to or interaction with a human alpha-1 antitrypsin (A1AT) protein, relative to that of the wild-type PPE peptidase domain. Particular examples of variants include a PPE peptidase domain that has at least one alteration at a residue selected from one or more of Q211, T55, D74, R75, S214, R237, and N241, the residue numbering being defined by SEQ ID NO: 1 (wild-type PPE proprotein). In specific embodiments, the at least one amino acid alteration is selected from one or more of Q211F, T55A, D74A, R75A, R75E, Q211A, S214A, R237A, N241A, and N241Y, the residue numbering being defined by SEQ ID NO: 1.

In certain embodiments, prior to cleavage, the modified PPE proprotein does not substantially bind to a serine protease inhibitor (Serpin) in vitro or in vivo, for example, wherein the Serpin is alpha-1 antitrypsin (A1AT). In some embodiments, prior to cleavage, the modified PPE proprotein is substantially inactive as a serine protease in its proprotein form.

In some embodiments, protease cleavage of the modified activation peptide, for example, at a cancer or tumor site in vivo, generates an active PPE peptidase domain (also referred to as an active PPE protein), which has increased serine protease activity relative to the modified PPE proprotein. In some embodiments, the serine protease activity of the active PPE protein is increased by about or at least about 2-fold, 5-fold, 10-fold, 50-fold, 100-fold, 500-fold, or 1000-fold or more relative to that of the modified PPE proprotein. In some embodiments, the active PPE protein has the same or substantially the same serine protease activity as a wild-type active PPE protein (e.g., SEQ ID NO: 7). In some embodiments, the active PPE protein is a variant that has about or at least about 50, 60, 70, 80, 90, 100, 200, 300, 400, 500, 600, 700, 800, 900, or 1000% or more of the serine protease activity of a wild-type active PPE protein (SEQ ID NO: 7).

In some embodiments, protease cleavage of the modified activation peptide, for example, at a cancer or tumor site in vivo, generates an active PPE peptidase domain (or active PPE protein), which has increased cancer cell-killing activity relative to the PPE proprotein. In certain embodiments, the cancer cell-killing activity of the active PPE protein is increased by about or at least about 2-fold, 5-fold, 10-fold, 50-fold, 100-fold, 500-fold, or 1000-fold or more relative to that of the PPE proprotein. In some embodiments, the active PPE protein has the same or substantially the same cancer cell-killing activity as a wild-type active PPE protein (e.g., SEQ ID NO: 7). In some embodiments, the active PPE protein is a variant that has about or at least about 50, 60, 70, 80, 90, 100, 200, 300, 400, 500, 600, 700, 800, 900, or 1000% or more of the cancer cell-killing activity of a wild-type active PPE protein (SEQ ID NO: 7).

Serine protease activity and cancer cell-killing activity can be measured according to routine techniques in the art. For example, serine protease activity can be monitored using a colorimetric substrate activity assay (N-Methoxysuccinyl-Ala-Ala-Pro-Val p-nitroanilide), and cancer cell-killing activity can be measured in vitro or in vivo.

### Methods of Use and Pharmaceutical Compositions

Certain embodiments include methods of treating, ameliorating the symptoms of, and/or reducing the progression of, a disease or condition in a subject in need thereof, comprising administering to the subject a composition comprising a modified serine protease proprotein, for example, a modified PPE proprotein, as described herein. In particular embodiments, the disease is a cancer, that is, the subject in need thereof has, is suspected of having, or is at risk for having, a cancer. As noted, the composition comprises a modified serine protease proprotein (inactive form), which is activated by cleavage of the modified activation peptide in a cancer tissue or tumor site of the subject in need thereof, to generate an active peptidase domain, or active protein.

In particular embodiments, the cancer is a primary cancer or a metastatic cancer. In specific embodiments, the cancer is selected from one or more of melanoma (optionally metastatic melanoma), breast cancer (optionally triple-negative breast cancer, TNBC), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer

In some embodiments, as noted above, the cancer is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Certain embodiments thus include combination therapies for treating cancers, including methods of treating ameliorating the symptoms of, or inhibiting the progression of, a cancer in a subject in need thereof, comprising administering to the subject a modified serine protease proprotein described herein in combination with at least one additional agent, for example, an immunotherapy agent, a chemotherapeutic agent, a hormonal therapeutic agent, and/or a kinase inhibitor. In some embodiments, administering the modified serine protease proprotein enhances the susceptibility of the cancer to the additional agent (for example, immunotherapy agent, chemotherapeutic agent, hormonal therapeutic agent, and or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone.

Certain combination therapies employ one or more cancer immunotherapy agents, or “immunotherapy agents”. In certain instances, an immunotherapy agent modulates the immune response of a subject, for example, to increase or maintain a cancer-related or cancer-specific immune response, and thereby results in increased immune cell inhibition or reduction of cancer cells. Exemplary immunotherapy agents include polypeptides, for example, antibodies and antigen-binding fragments thereof, ligands, and small peptides, and mixtures thereof. Also include as immunotherapy agents are small molecules, cells (e.g., immune cells such as T-cells), various cancer vaccines, gene therapy or other polynucleotide-based agents, including viral agents such as oncolytic viruses, and others known in the art. Thus, in certain embodiments, the cancer immunotherapy agent is selected from one or more of immune checkpoint modulatory agents, cancer vaccines, oncolytic viruses, cytokines, and cell-based immunotherapies.

In certain embodiments, the cancer immunotherapy agent is an immune checkpoint modulatory agent. Particular examples include “antagonists” of one or more inhibitory immune checkpoint molecules, and “agonists” of one or more stimulatory immune checkpoint molecules. Generally, immune checkpoint molecules are components of the immune system that either turn up a signal (co-stimulatory molecules) or turn down a signal, the targeting of which has therapeutic potential in cancer because cancer cells can perturb the natural function of immune checkpoint molecules (see, e.g., Sharma and Allison, Science. 348:56-61, 2015; Topalian et al., Cancer Cell. 27:450-461, 2015; Pardoll, Nature Reviews Cancer. 12:252-264, 2012). In some embodiments, the immune checkpoint modulatory agent (e.g., antagonist, agonist) “binds” or “specifically binds” to the one or more immune checkpoint molecules, as described herein.

In some embodiments, the immune checkpoint modulatory agent is an antagonist or inhibitor of one or more inhibitory immune checkpoint molecules. Exemplary inhibitory immune checkpoint molecules include Programmed Death-Ligand 1 (PD-L1), Programmed Death-Ligand 2 (PD-L2), Programmed Death 1 (PD-1), V-domain Ig suppressor of T cell activation (VISTA), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), B and T Lymphocyte Attenuator (BTLA), CD160, and T-cell immunoreceptor with Ig and ITIM domains (TIGIT).

In certain embodiments, the agent is a PD-1 (receptor) antagonist or inhibitor, the targeting of which has been shown to restore immune function in the tumor environment (see, e.g., Phillips et al., Int Immunol. 27:39-46, 2015). PD-1 is a cell surface receptor that belongs to the immunoglobulin superfamily and is expressed on T cells and pro-B cells. PD-1 interacts with two ligands, PD-L1 and PD-L2. PD-1 functions as an inhibitory immune checkpoint molecule, for example, by reducing or preventing the activation of T-cells, which in turn reduces autoimmunity and promotes self-tolerance. The inhibitory effect of PD-1 is accomplished at least in part through a dual mechanism of promoting apoptosis in antigen specific T-cells in lymph nodes while also reducing apoptosis in regulatory T cells (suppressor T cells). Some examples of PD-1 antagonists or inhibitors include an antibody or antigen-binding fragment or small molecule that specifically binds to PD-1 and reduces one or more of its immune-suppressive activities, for example, its downstream signaling or its interaction with PD-L1. Specific examples of PD-1 antagonists or inhibitors include the antibodies nivolumab, pembrolizumab, PDR001, MK-3475, AMP-224, AMP-514, and pidilizumab, and antigen-binding fragments thereof (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579).

In some embodiments, the agent is a PD-L1 antagonist or inhibitor. As noted above, PD-L1 is one of the natural ligands for the PD-1 receptor. General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MEDI4736), and antigen-binding fragments thereof (see, e.g., U.S. Pat. Nos. 9,102,725; 9,393,301; 9,402,899; 9,439,962).

In some embodiments, the agent is a PD-L2 antagonist or inhibitor. As noted above, PD-L2 is one of the natural ligands for the PD-1 receptor. General examples of PD-L2 antagonists or inhibitors include an antibody or antigen-binding fragment or small molecule that specifically binds to PD-L2 and reduces one or more of its immune-suppressive activities, for example, its binding to the PD-1 receptor.

In certain embodiments, the agent is a VISTA antagonist or inhibitor. VISTA is approximately 50 kDa in size and belongs to the immunoglobulin superfamily (it has one IgV domain) and the B7 family. It is primarily expressed in white blood cells, and its transcription is partially controlled by p53. There is evidence that VISTA can act as both a ligand and a receptor on T cells to inhibit T cell effector function and maintain peripheral tolerance. VISTA is produced at high levels in tumor-infiltrating lymphocytes, such as myeloid-derived suppressor cells and regulatory T cells, and its blockade with an antibody results in delayed tumor growth in mouse models of melanoma and squamous cell carcinoma. Exemplary anti-VISTA antagonist antibodies include, for example, the antibodies described in WO 2018/237287, which is incorporated by reference in its entirety.

In some embodiments, the agent is a CTLA-4 antagonist or inhibitor. CTLA4 or CTLA-4 (cytotoxic T-lymphocyte-associated protein 4), also known as CD152 (cluster of differentiation 152), is a protein receptor that functions as an inhibitory immune checkpoint molecule, for example, by transmitting inhibitory signals to T-cells when it is bound to CD80 or CD86 on the surface of antigen-presenting cells. General examples CTLA-4 antagonists or inhibitors include an antibody or antigen-binding fragment or small molecule that specifically binds to CTLA-4. Particular examples include the antibodies ipilimumab and tremelimumab, and antigen-binding fragments thereof. At least some of the activity of ipilimumab is believed to be mediated by antibody-dependent cell-mediated cytotoxicity (ADCC) killing of suppressor Tregs that express CTLA-4.

In some embodiments, the agent is an IDO antagonist or inhibitor, or a TDO antagonist or inhibitor. IDO and TDO are tryptophan catabolic enzymes with immune-inhibitory properties. For example, IDO is known to suppress T-cells and NK cells, generate and activate Tregs and myeloid-derived suppressor cells, and promote tumor angiogenesis. General examples of IDO and TDO antagonists or inhibitors include an antibody or antigen-binding fragment or small molecule that specifically binds to IDO or TDO (see, e.g., Platten et al., Front Immunol. 5: 673, 2014) and reduces or inhibits one or more immune-suppressive activities. Specific examples of IDO antagonists or inhibitors include indoximod (NLG-8189), 1-methyl-tryptophan (1MT), β-Carboline (norharmane; 9H-pyrido[3,4-b]indole), rosmarinic acid, and epacadostat (see, e.g., Sheridan, Nature Biotechnology. 33:321-322, 2015). Specific examples of TDO antagonists or inhibitors include 680C91 and LM10 (see, e.g., Pilotte et al., PNAS USA. 109:2497-2502, 2012).

In some embodiments, the agent is a TIM-3 antagonist or inhibitor. T-cell Immunoglobulin domain and Mucin domain 3 (TIM-3) is expressed on activated human CD4+ T-cells and regulates Th1 and Th17 cytokines. TIM-3 also acts as a negative regulator of Th1/Tc1 function by triggering cell death upon interaction with its ligand, galectin-9. TIM-3 contributes to the suppressive tumor microenvironment and its overexpression is associated with poor prognosis in a variety of cancers (see, e.g., Li et al., Acta Oncol. 54:1706-13, 2015). General examples of TIM-3 antagonists or inhibitors include an antibody or antigen-binding fragment or small molecule that specifically binds to TIM-3 and reduces or inhibits one or more of its immune-suppressive activities.

In some embodiments, the agent is a LAG-3 antagonist or inhibitor. Lymphocyte Activation Gene-3 (LAG-3) is expressed on activated T-cells, natural killer cells, B-cells and plasmacytoid dendritic cells. It negatively regulates cellular proliferation, activation, and homeostasis of T-cells, in a similar fashion to CTLA-4 and PD-1 (see, e.g., Workman and Vignali. European Journal of Immun. 33: 970-9, 2003; and Workman et al., Journal of Immun. 172: 5450-5, 2004), and has been reported to play a role in Treg suppressive function (see, e.g., Huang et al., Immunity. 21: 503-13, 2004). LAG3 also maintains CD8+ T-cells in a tolerogenic state and combines with PD-1 to maintain CD8 T-cell exhaustion. General examples of LAG-3 antagonists or inhibitors include an antibody or antigen-binding fragment or small molecule that specifically binds to LAG-3 and inhibits one or more of its immune-suppressive activities. Specific examples include the antibody BMS-986016, and antigen-binding fragments thereof.

In some embodiments, the agent is a BTLA antagonist or inhibitor. B- and T-lymphocyte attenuator (BTLA; CD272) expression is induced during activation of T-cells, and it inhibits T-cells via interaction with tumor necrosis family receptors (TNF-R) and B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses, for example, by inhibiting the function of human CD8+ cancer-specific T-cells (see, e.g., Derré et al., J Clin Invest 120:157-67, 2009). General examples of BTLA antagonists or inhibitors include an antibody or antigen-binding fragment or small molecule that specifically binds to BTLA-4 and reduce one or more of its immune-suppressive activities.

In some embodiments, the agent is an HVEM antagonist or inhibitor, for example, an antagonist or inhibitor that specifically binds to HVEM and interferes with its interaction with BTLA or CD160. General examples of HVEM antagonists or inhibitors include an antibody or antigen-binding fragment or small molecule that specifically binds to HVEM, optionally reduces the HVEM/BTLA and/or HVEM/CD160 interaction, and thereby reduces one or more of the immune-suppressive activities of HVEM.

In some embodiments, the agent is a CD160 antagonist or inhibitor, for example, an antagonist or inhibitor that specifically binds to CD160 and interferes with its interaction with HVEM. General examples of CD160 antagonists or inhibitors include an antibody or antigen-binding fragment or small molecule that specifically binds to CD160, optionally reduces the CD160/HVEM interaction, and thereby reduces or inhibits one or more of its immune-suppressive activities.

In some embodiments, the agent is a TIGIT antagonist or inhibitor. T cell Ig and ITIM domain (TIGIT) is a co-inhibitory receptor that is found on the surface of a variety of lymphoid cells, and suppresses antitumor immunity, for example, via Tregs (Kurtulus et al., J Clin Invest. 125:4053-4062, 2015). General examples of TIGIT antagonists or inhibitors include an antibody or antigen-binding fragment or small molecule that specifically binds to TIGIT and reduce one or more of its immune-suppressive activities (see, e.g., Johnston et al., Cancer Cell. 26:923-37, 2014).

In certain embodiments, the immune checkpoint modulatory agent is an agonist of one or more stimulatory immune checkpoint molecules. Exemplary stimulatory immune checkpoint molecules include CD40, OX40, Glucocorticoid-Induced TNFR Family Related Gene (GITR), CD137 (4-1BB), CD27, CD28, CD226, and Herpes Virus Entry Mediator (HVEM).

In some embodiments, the agent is a CD40 agonist. CD40 is expressed on antigen-presenting cells (APC) and some malignancies. Its ligand is CD40L (CD154). On APC, ligation results in upregulation of costimulatory molecules, potentially bypassing the need for T-cell assistance in an antitumor immune response. CD40 agonist therapy plays an important role in APC maturation and their migration from the tumor to the lymph nodes, resulting in elevated antigen presentation and T cell activation. Anti-CD40 agonist antibodies produce substantial responses and durable anticancer immunity in animal models, an effect mediated at least in part by cytotoxic T-cells (see, e.g., Johnson et al. Clin Cancer Res. 21: 1321-1328, 2015; and Vonderheide and Glennie, Clin Cancer Res. 19:1035-43, 2013). General examples of CD40 agonists include an antibody or antigen-binding fragment or small molecule or ligand that specifically binds to CD40 and increases one or more of its immunostimulatory activities. Specific examples include CP-870,893, dacetuzumab, Chi Lob 7/4, ADC-1013, CD40L, rhCD40L, and antigen-binding fragments thereof. Specific examples of CD40 agonists include, but are not limited to, APX005 (see, e.g., US 2012/0301488) and APX005M (see, e.g., US 2014/0120103).

In some embodiments, the agent is an OX40 agonist. OX40 (CD134) promotes the expansion of effector and memory T cells, and suppresses the differentiation and activity of T-regulatory cells (see, e.g., Croft et al., Immunol Rev. 229:173-91, 2009). Its ligand is OX40L (CD252). Since OX40 signaling influences both T-cell activation and survival, it plays a key role in the initiation of an anti-tumor immune response in the lymph node and in the maintenance of the anti-tumor immune response in the tumor microenvironment. General examples of OX40 agonists include an antibody or antigen-binding fragment or small molecule or ligand that specifically binds to OX40 and increases one or more of its immunostimulatory activities. Specific examples include OX86, OX-40L, Fc-OX40L, GSK3174998, MEDI0562 (a humanized OX40 agonist), MEDI6469 (murine OX4 agonist), and MEDI6383 (an OX40 agonist), and antigen-binding fragments thereof.

In some embodiments, the agent is a GITR agonist. Glucocorticoid-Induced TNFR family Related gene (GITR) increases T cell expansion, inhibits the suppressive activity of Tregs, and extends the survival of T-effector cells. GITR agonists have been shown to promote an anti-tumor response through loss of Treg lineage stability (see, e.g., Schaer et al., Cancer Immunol Res. 1:320-31, 2013). These diverse mechanisms show that GITR plays an important role in initiating the immune response in the lymph nodes and in maintaining the immune response in the tumor tissue. Its ligand is GITRL. General examples of GITR agonists include an antibody or antigen-binding fragment or small molecule or ligand that specifically binds to GITR and increases one or more of its immunostimulatory activities. Specific examples include GITRL, INCAGN01876, DTA-1, MEDI1873, and antigen-binding fragments thereof.

In some embodiments, the agent is a CD137 agonist. CD137 (4-1BB) is a member of the tumor necrosis factor (TNF) receptor family, and crosslinking of CD137 enhances T-cell proliferation, IL-2 secretion, survival, and cytolytic activity. CD137-mediated signaling also protects T-cells such as CD8+ T-cells from activation-induced cell death. General examples of CD137 agonists include an antibody or antigen-binding fragment or small molecule or ligand that specifically binds to CD137 and increases one or more of its immunostimulatory activities. Specific examples include the CD137 (or 4-1BB) ligand (see, e.g., Shao and Schwarz, J Leukoc Biol. 89:21-9, 2011) and the antibody utomilumab, including antigen-binding fragments thereof.

In some embodiments, the agent is a CD27 agonist. Stimulation of CD27 increases antigen-specific expansion of naïve T cells and contributes to T-cell memory and long-term maintenance of T-cell immunity. Its ligand is CD70. The targeting of human CD27 with an agonist antibody stimulates T-cell activation and antitumor immunity (see, e.g., Thomas et al., Oncoimmunology. 2014;3:e27255. doi:10.4161/onci.27255; and He et al., J Immunol. 191:4174-83, 2013). General examples of CD27 agonists include an antibody or antigen-binding fragment or small molecule or ligand that specifically binds to CD27 and increases one or more of its immunostimulatory activities. Specific examples include CD70 and the antibodies varlilumab and CDX-1127 (1F5), including antigen-binding fragments thereof.

In some embodiments, the agent is a CD28 agonist. CD28 is constitutively expressed CD4+ T cells some CD8+ T cells. Its ligands include CD80 and CD86, and its stimulation increases T-cell expansion. General examples of CD28 agonists include an antibody or antigen-binding fragment or small molecule or ligand that specifically binds to CD28 and increases one or more of its immunostimulatory activities. Specific examples include CD80, CD86, the antibody TAB08, and antigen-binding fragments thereof.

In some embodiments, the agent is CD226 agonist. CD226 is a stimulating receptor that shares ligands with TIGIT, and opposite to TIGIT, engagement of CD226 enhances T-cell activation (see, e.g., Kurtulus et al., J Clin Invest. 125:4053-4062, 2015; Bottino et al., J Exp Med. 1984:557-567, 2003; and Tahara-Hanaoka et al., Int Immunol. 16:533-538, 2004). General examples of CD226 agonists include an antibody or antigen-binding fragment or small molecule or ligand (e.g., CD112, CD155) that specifically binds to CD226 and increases one or more of its immunostimulatory activities.

In some embodiments, the agent is an HVEM agonist. Herpesvirus entry mediator (HVEM), also known as tumor necrosis factor receptor superfamily member 14 (TNFRSF14), is a human cell surface receptor of the TNF-receptor superfamily. HVEM is found on a variety of cells including T-cells, APCs, and other immune cells. Unlike other receptors, HVEM is expressed at high levels on resting T-cells and down-regulated upon activation. It has been shown that HVEM signaling plays a crucial role in the early phases of T-cell activation and during the expansion of tumor-specific lymphocyte populations in the lymph nodes. General examples of HVEM agonists include an antibody or antigen-binding fragment or small molecule or ligand that specifically binds to HVEM and increases one or more of its immunostimulatory activities.

In certain embodiments, the immunotherapy agent is a bi-specific or multi-specific antibody. For instance, certain bi-specific or multi-specific antibodies are able to (i) bind to and inhibit one or more inhibitory immune checkpoint molecules, and also (ii) bind to and agonize one or more stimulatory immune checkpoint molecules. In certain embodiments, a bi-specific or multi-specific antibody (i) binds to and inhibits one or more of PD-L1, PD-L2, PD-1, CTLA-4, IDO, TDO, TIM-3, LAG-3, BTLA, CD160, and/or TIGIT, and also (ii) binds to and agonizes one or more of CD40, OX40 Glucocorticoid-Induced TNFR Family Related Gene (GITR), CD137 (4-1BB), CD27, CD28, CD226, and/or Herpes Virus Entry Mediator (HVEM).

In some embodiments, the immunotherapy agent is a cancer vaccine. In certain embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In some embodiments, the immunotherapy agent is an oncolytic viruses. In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

In certain embodiments, the cancer immunotherapy agent is a cytokine. Exemplary cytokines include interferon (IFN)-α, IL-2, IL-12, IL-7, IL-21, and Granulocyte-macrophage colony-stimulating factor (GM-CSF).

In certain embodiments, the cancer immunotherapy agent is cell-based immunotherapy, for example, a therapy that utilizes immune cells, including ex vivo-derived immune cells, such as lymphocytes, natural killer (NK) cells, macrophages, and/or dendritic cells (DCs). In some embodiments, the lymphocytes comprise T-cells, for example, cytotoxic T-lymphocytes (CTLs). See, for example, June, J Clin Invest. 117: 1466-1476, 2007; Rosenberg and Restifo, Science. 348:62-68, 2015; Cooley et al., Biol. of Blood and Marrow Transplant. 13:33-42, 2007; and Li and Sun, Chin J Cancer Res. 30:173-196, 2018, for descriptions of adoptive T-cell and NK cell immunotherapies. In some embodiments, the T-cells comprise cancer antigen-specific T-cells, which are directed against at least one cancer antigen. In some embodiments, the cancer antigen-specific T-cells are selected from one or more of chimeric antigen receptor (CAR)-modified T-cells, T-cell Receptor (TCR)-modified T-cells, tumor infiltrating lymphocytes (TILs), and peptide-induced T-cells. In specific embodiments, the CAR-modified T-cell is targeted against CD-19 (see, e.g., Maude et al., Blood. 125:4017-4023, 2015). In some instances, the ex vivo-derived immune cells are autologous cells, which are obtained from the patient to be treated.

Certain combination therapies employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type 1 or type II), and anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The various chemotherapeutic agents described herein can be combined with any one or more of the modified serine protease proproteins described herein, and used according to any one or more of the methods or compositions described herein.

Certain combination therapies employ at least one hormonal therapeutic agent. General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

The various hormonal therapeutic agents described herein can be combined with any one or more of the modified serine protease proproteins described herein, and used according to any one or more of the methods or compositions described herein.

Certain combination therapies employ at least one kinase inhibitor, including tyrosine kinase inhibitors. Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

The various kinase inhibitors described herein can be combined with any one or more of the modified serine protease proproteins described herein, and used according to any one or more of the methods or compositions described herein.

In some embodiments, the methods and compositions described herein increase cancer cell-killing in the subject by about or at least about 2-fold, 5-fold, 10-fold, 50-fold, 100-fold, 500-fold, or 1000-fold or more relative to a control or reference. In some embodiments, the methods and compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and compositions described herein increase median survival time of a subject by 1 year, 2 years, 3 years, or longer. In some embodiments, the methods and pharmaceutical compositions increase progression-free survival by 2 weeks, 3 weeks, 4 weeks, 5 weeks, 6 weeks, 7 weeks, 8 weeks, 9 weeks, 10 weeks or longer. In certain embodiments, the methods and pharmaceutical compositions described herein increase progression-free survival by 1 year, 2 years, 3 years, or longer.

In certain embodiments, the methods and compositions described herein are sufficient to result in tumor regression, for example, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and compositions described herein are sufficient to result in stable disease. In certain embodiments, the methods and compositions described herein are sufficient to result in clinically relevant reduction in symptoms of a particular disease indication known to the skilled clinician.

For in vivo use, as noted above, for the treatment of human or non-human mammalian disease or testing, the modified serine protease proproteins described herein are generally incorporated into one or more therapeutic or pharmaceutical compositions prior to administration, including veterinary therapeutic compositions.

Thus, certain embodiments relate to pharmaceutical or therapeutic compositions that comprise a modified serine protease proprotein, as described herein. In some instances, a pharmaceutical or therapeutic composition comprises one or more of the modified serine protease proproteins described herein in combination with a pharmaceutically- or physiologically-acceptable carrier or excipient. Certain pharmaceutical or therapeutic compositions further comprise at least one additional agent, for example, an immunotherapy agent, a chemotherapeutic agent, a hormonal therapeutic agent, and/or a kinase inhibitor as described herein.

Some therapeutic compositions comprise (and certain methods utilize) only one modified serine protease proprotein. Certain therapeutic compositions comprise (and certain methods utilize) a mixture of at least two, three, four, or five different modified serine protease proproteins.

In particular embodiments, the pharmaceutical or therapeutic compositions comprising a modified serine protease proprotein is substantially pure on a protein basis or a weight-weight basis, for example, the composition has a purity of at least about 80%, 85%, 90%, 95%, 98%, or 99% on a protein basis or a weight-weight basis.

In some embodiments, the modified serine protease proproteins described herein do not form aggregates, have a desired solubility, and/or have an immunogenicity profile that is suitable for use in humans, as known in the art. Thus, in some embodiments, the therapeutic composition comprising a modified serine protease proprotein is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2 % high molecular weight aggregated proteins, or less than about 1% high molecular weight aggregated proteins. Some compositions comprise a modified serine protease proprotein that is at least about 50%, about 60%, about 70%, about 80%, about 90% or about 95% monodisperse with respect to its apparent molecular mass.

In some embodiments, the modified serine protease proproteins are concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11, 12, 13, 14 or 15 mg/ml and are formulated for biotherapeutic uses.

To prepare a therapeutic or pharmaceutical composition, an effective or desired amount of one or more modified serine protease proproteins is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of modified serine protease proproteins described herein, in pure form or in an appropriate therapeutic or pharmaceutical composition, can be carried out via any of the accepted modes of administration of agents for serving similar utilities. The therapeutic or pharmaceutical compositions can be prepared by combining a modified serine protease proprotein-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous, or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington’s Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer’s solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

The therapeutic or pharmaceutical compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In some instances, a therapeutically effective daily dose is (for a 70 kg mammal) from about 0.001 mg/kg (i.e., ~ 0.07 mg) to about 100 mg/kg (i.e., ~ 7.0 g); preferably a therapeutically effective dose is (for a 70 kg mammal) from about 0.01 mg/kg (i.e., ~ 0.7 mg) to about 50 mg/kg (i.e., ~ 3.5 g); more preferably a therapeutically effective dose is (for a 70 kg mammal) from about 1 mg/kg (i.e., ~ 70 mg) to about 25 mg/kg (i.e., ~ 1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains a modified serine protease proprotein and an additional therapeutic agent (e.g., immunotherapy agent, chemotherapeutic agent, hormonal therapeutic agent, kinase inhibitor), as well as administration of compositions comprising a modified serine protease proprotein and an additional therapeutic agent in its own separate pharmaceutical dosage formulation. For example, a modified serine protease proprotein and an additional therapeutic agent can be administered to the subject together in a single parenteral dosage composition such as in a saline solution or other physiologically acceptable solution, or each agent administered in separate parenteral dosage formulations. Where separate dosage formulations are used, the compositions can be administered at essentially the same time, i.e., concurrently, or at separately staggered times, i.e., sequentially and in any order; combination therapy is understood to include all these regimens.

Also included are patient care kits, comprising (a) at least one modified serine protease proprotein, as described herein; and optionally (b) at least one additional therapeutic agent (e.g., immunotherapy agent, chemotherapeutic agent, hormonal therapeutic agent, kinase inhibitor). In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a modified serine protease proprotein and optionally at least one additional therapeutic agent. For example, the kit includes a plurality of syringes, ampules, foil packets, or blister packs, each containing a single unit dose of a modified serine protease proprotein and optionally at least one additional therapeutic agent. The containers of the kits can be air tight, waterproof (e.g., impermeable to changes in moisture or evaporation), and/or light-tight.

The patient care kit optionally includes a device suitable for administration of the composition, e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

### Expression and Purification Systems

Certain embodiments include methods and related compositions for expressing and purifying a modified serine protease proprotein described herein. Such recombinant modified serine protease proproteins can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, a modified serine protease proprotein may be prepared by a procedure including one or more of the steps of: (a) preparing a vector or construct comprising a polynucleotide sequence that encodes a modified serine protease proprotein described herein (see, e.g., Table S4), which is operably linked to one or more regulatory elements; (b) introducing the vector or construct into a host cell; (c) culturing the host cell to express the modified serine protease proprotein; and (d) isolating the modified serine protease proprotein from the host cell.

To express a desired polypeptide, a nucleotide sequence encoding a modified serine protease proprotein may be inserted into appropriate expression vector(s), i.e., vector(s) which contain the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector--enhancers, promoters, 5′ and 3′ untranslated regions--which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21(DE3), a λDE3 lysogen of BL21 that supports T7-mediated expression and is deficient in Ion and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS•TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif. 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210 - 215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409 -1414, 2004; U.S. Pat. Nos. 7,629,163; 7,326,681; and 7,029,872). Merely by way of example, recombinant yeast cultures can be grown in Fernbach Flasks or 15L, 50L, 100L, and 200L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., Proc. Natl. Acad. Sci. U.S.A. 91:3224-3227 (1994)). Also included are baculovirus expression systems, including those that utilize SF9, SF21, and T. ni cells (see, e.g., Murphy and Piwnica-Worms, Curr Protoc Protein Sci. Chapter 5:Unit5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for protein production, see, e.g., Yazaki and Wu, Methods in Molecular Biology, Vol. 248 (B. K.C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1L and 5L spinners, 5L, 14L, 40L, 100L and 200L stir tank bioreactors, or 20/50L and 100/200L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt- cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009).

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with one or more polynucleotide sequences of interest may be cultured under conditions suitable for the expression and recovery of the protein from cell culture. Certain specific embodiments utilize serum free cell expression systems. Examples include HEK293 cells and CHO cells that can grow in serum free medium (see, e.g., Rosser et al., Protein Expr. Purif. 40:237-43, 2005; and U.S. Pat. number 6,210,922).

A modified serine protease proprotein produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin, FLAG tags, poly-histidine tags (e.g., 6xHis), cMyc tags, V5-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-performance liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

Also included are methods of concentrating a modified serine protease proprotein, and compositions comprising concentrated soluble a modified serine protease proprotein. In some aspects, such concentrated solutions of a modified serine protease proprotein comprises proteins at a concentration of about or at least about 5 mg/mL, 8 mg/mL, 10 mg/mL, 15 mg/mL, 20 mg/mL, or more.

In some aspects, such compositions may be substantially monodisperse, meaning that a modified serine protease proprotein exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, a modified serine protease proprotein in a composition has a purity of at least about 90%, as measured according to routine techniques in the art. In certain embodiments, such as diagnostic compositions or certain pharmaceutical or therapeutic compositions, a modified serine protease proprotein in a composition has a purity of at least about 95%, or at least about 97% or 98% or 99%. In some embodiments, such as when being used as reference or research reagents, a modified serine protease proprotein can be of lesser purity, and may have a purity of at least about 50%, 60%, 70%, or 80%. Purity can be measured overall or in relation to selected components, such as other proteins, e.g., purity on a protein basis.

Purified modified serine protease proproteins can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more biological activities can be measured according to in vitro or cell-based assays, which are optionally functionally coupled to a readout or indicator, such as a fluorescent or luminescent indicator of biological activity, as described herein.

In certain embodiments, as noted above, a composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, a modified serine protease proprotein is made from a eukaryotic cell such as a mammalian or human cell in substantially serum free media. In certain embodiments, as noted herein, a composition has an endotoxin content of less than about 10 EU/mg of protein, or less than about 5 EU/mg of protein, less than about 3 EU/mg of protein, or less than about 1 EU/mg of protein.

In certain embodiments, a composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N— and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade modified serine protease proproteins can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of a modified serine protease proprotein and variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. A modified serine protease proprotein with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains a modified serine protease proprotein of the present disclosure.

Although the foregoing embodiments have been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Activity and Binding Characteristics of PPE

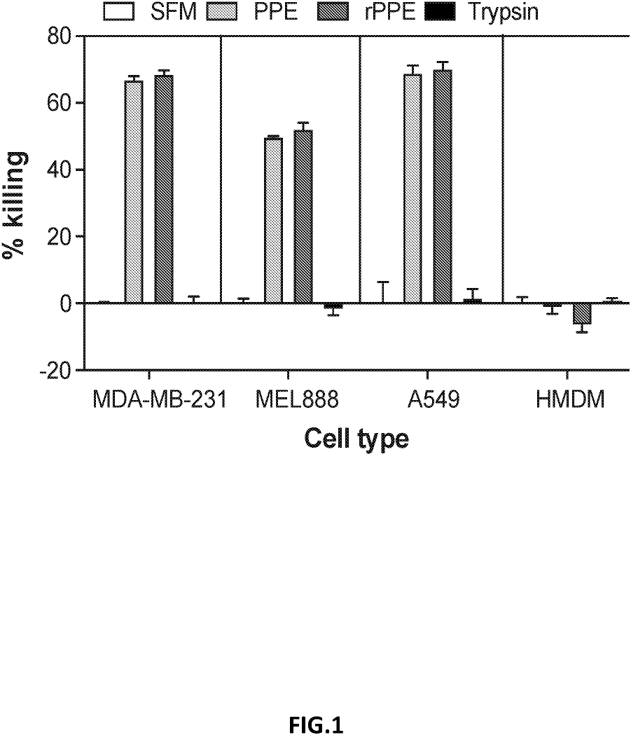

To test the cancer-cell killing activity of PPE, wild-type recombinant PPE proprotein (pro-rPPE) was activated by incubation with 1/20 (w/w) trypsin for 2 hours at 37° C. to generate an active PPE protein (rPPE). Human cancer cells (MDA-MB-231, a triple-negative breast cancer (TNBC) cell line; MEL888, a melanoma cell line; and A549, a lung adenocarcinoma cell line) and non-cancer cells (HMDMs, or human monocyte-derived macrophages isolated from healthy donors) were treated with serum-free media (SFM) in the presence or absence of rPPE (50 nM), native PPE (50 nM), or trypsin (1:20 w/w; control for presence of activating trypsin) for 6 hours. Cell-killing was quantified by calcein-AM. As shown in FIG. 1, recombinant and native forms of activated PPE are able to selectively kill a variety of cancer cells, but are non-toxic to normal or non-cancer cells.

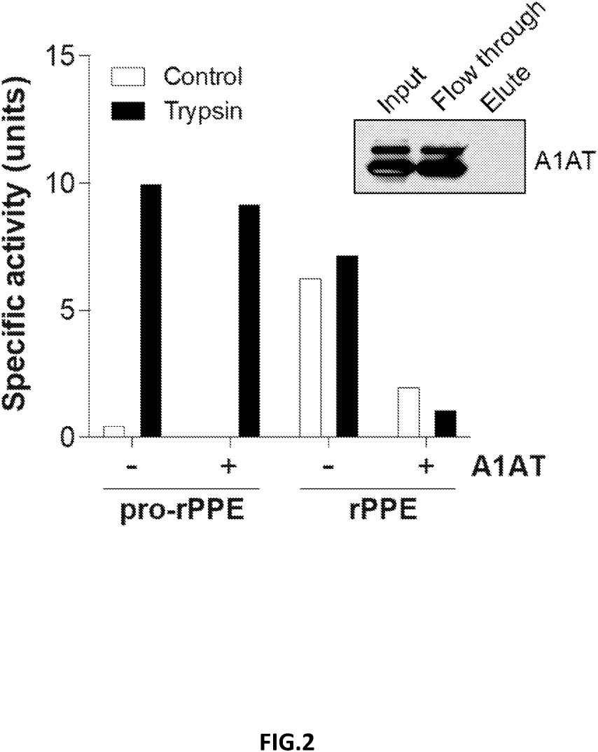

To test binding to the Serpin A1AT, the wild-type PPE proprotein (pro-rPPE) with a C-terminal his-tag was incubated in the presence/absence of a 20-fold excess of A1AT for 30 minutes at room temperature. After incubation, pro-rPPE was purified by Ni-Sepharose beads. Purified pro-rPPE was then treated with trypsin for 2 hours at 37° C. to activate the enzyme, and catalytic activity was assessed using a colorimetric activity assay. As a control, pro-rPPE was first activated with trypsin to generate active rPPE and then taken through an identical A1AT-binding/purification procedure. The results in FIG. 2 show full recovery of protease activity for pro-rPPE following isolation from the A1AT solution, suggesting that pro-rPPE does not bind to A1AT. In contrast, the protease activity of activated rPPE was attenuated by A1AT when subjected to an identical procedure. The FIG. 2 inset shows immunoblotting for A1AT pre- and post-purification of pro-rPPE.

### Example 2

### Engineering and Testing of Modified PPE Proproteins

Modified PPE proproteins (see Table S4) were engineered to comprise modified activation peptides (see Table S3) and expressed as N-terminal His-tagged proteins in CHO cells. For example, “Mutant 2” is a PPE proprotein that comprises the modified activation peptide of SEQ ID NO: 9 (MMP12 cleavage site, PPE optimized), and “Mutant 3” is a PPE proprotein that comprises the modified activation peptide of SEQ ID NO: 10 (MMP12 cleavage site, balanced).

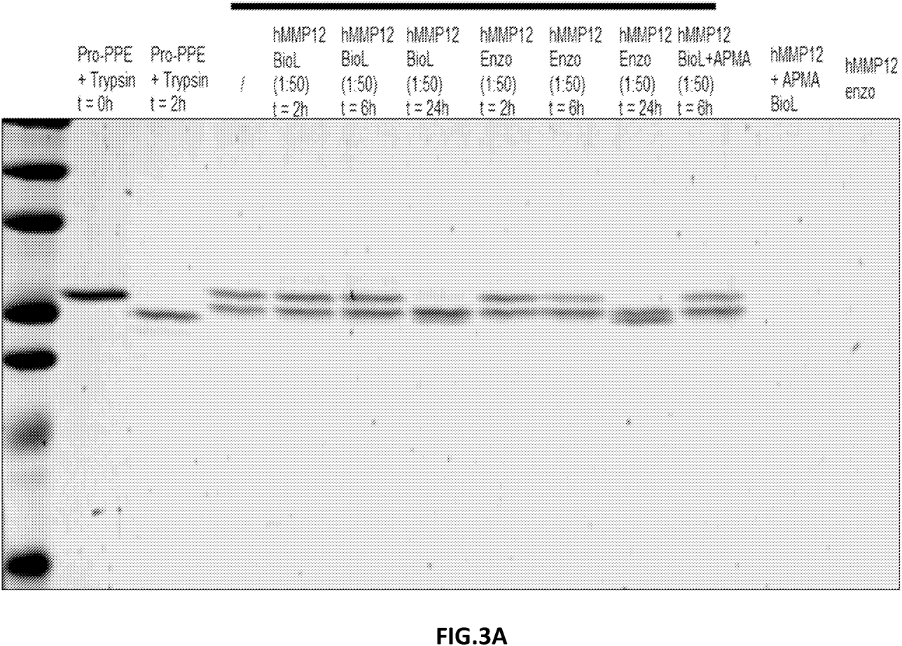

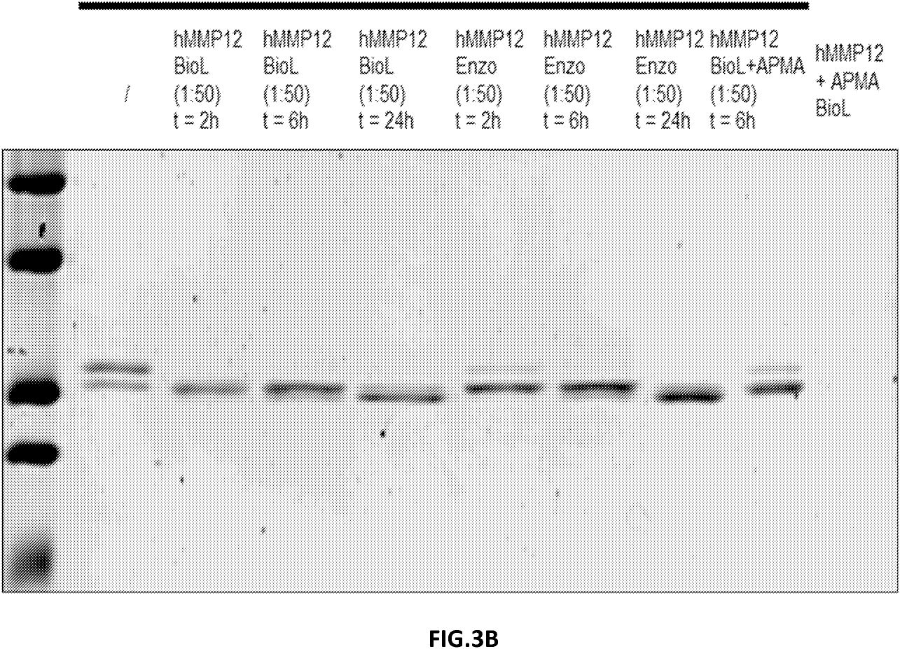

To test protease cleavage, wild-type PPE and modified PPE proproteins were incubated in vitro at 37° C. with purified trypsin or human MMP12 (BioLegend (BioL) or Enzo Life Sciences (Enzo); 1/50 w/w) for the indicated times, and evaluated by SDS-PAGE and Coomassie blue staining for evidence of protease cleavage to produce active PPE peptidase domains (or active PPE proteins). FIGS. 3A-3B show that the MMP12 protease cleaved the exemplary modified PPE proteins designated as Mutant 2 (3A) and Mutant 3 (3B). FIG. 3A also shows that trypsin cleaved the wild-type PPE as a control.

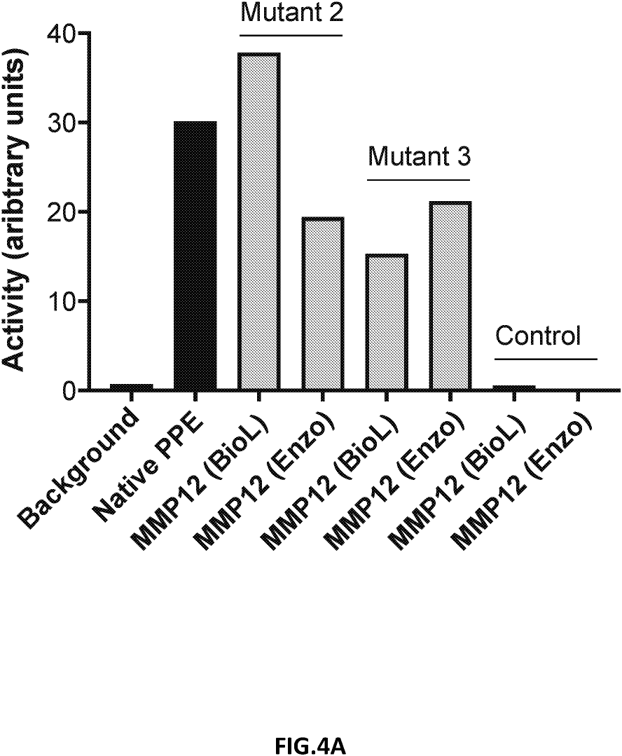

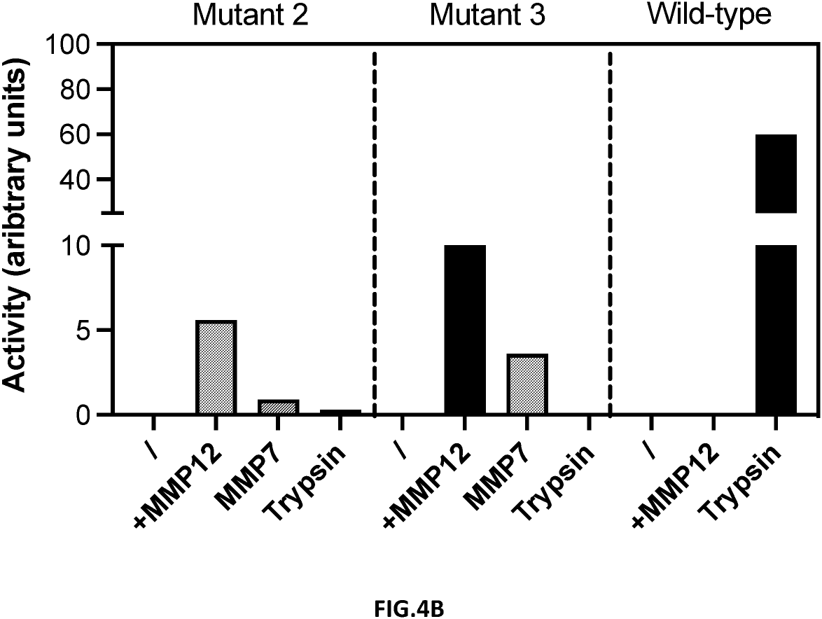

Wild-type PPE and modified PPE proproteins were tested in vitro for enzyme activity following protease activation. In one set of experiments, protease digestion was performed for 24 hours at 37° C. with MMP12 (BioLegend (BioL) or Enzo Life Sciences (Enzo); 1/50 w/w), and catalytic or enzymatic activity was monitored using a colorimetric substrate activity assay (N-Methoxysuccinyl-Ala-Ala-Pro-Val p-nitroanilide; Millipore Sigma). FIG. 4A shows that the exemplary modified PPE proteins were catalytically-active after incubation by MMP12, relative to the activity of the native PPE as a control. In another set of experiments, protease digestion was performed with trypsin (1/50, w/w, 2h, 37° C.), human MMP12 (Enzo, 1/50, w/w, 24h, 37° C.), and human MMP7 (Millipore Sigma, 1/50, w/w, 24h, 37° C.), and catalytic activity was assessed as above. FIG. 4B shows that the exemplary modified PPE proproteins were catalytically-active after incubation with MMP12, partially-catalytically-active after incubation with MMP7, and catalytically-inactive after incubation with trypsin or no protease. In contrast, the wild-type PPE protein was catalytically-active after incubation with trypsin, and catalytically-inactive after incubation with MMP12 or no protease.

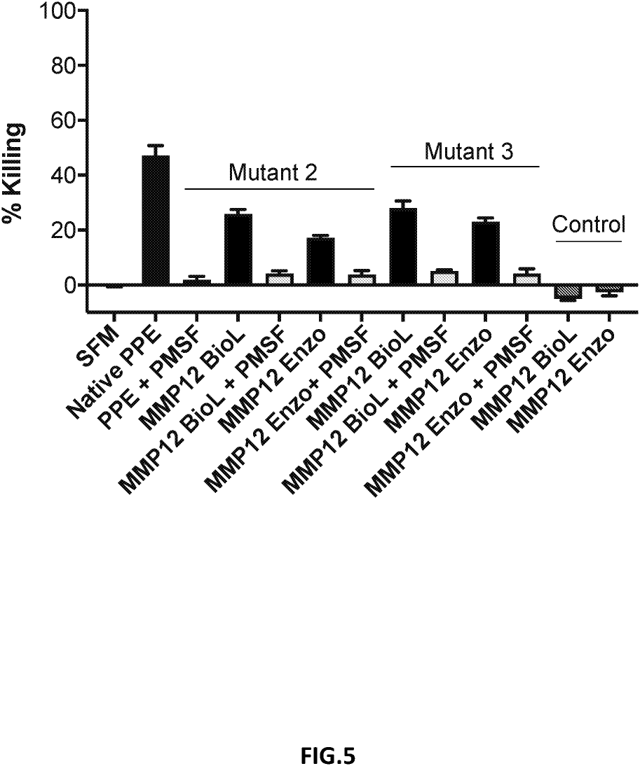

The modified PPE proproteins were then tested in vitro for cancer cell-killing activity. Cancer cell-killing activity was assessed by incubating human cancer cells (MDA-MB-231) with MMP12 protease-treated test proteins in serum-free media for about 12 hours and measuring cell viability by calcein-AM. Native PPE and MMP12 enzymes alone were included as controls. As shown in FIG. 5, the exemplary modified PPE proteins showed significant cancer cell-killing activity after incubation with (and activation by) the MMP12 protease.

The activity of modified PPE proproteins is tested in vivo by intratumoral injection into cancerous mice, representing various cancer models. Tumor growth, cancer cell apoptosis, and immune cell responses are monitored. Candidates that show efficacy in intratumoral delivery models are re-tested in vivo by intravenous injection into the cancerous mice.

1. A modified serine protease proprotein, comprising in an N-terminal to C-terminal orientation, a signal peptide, a modified activation peptide, and a peptidase domain, wherein the modified activation peptide comprises a heterologous protease cleavage site that is cleavable by a protease selected from a metalloprotease, an aspartyl protease, and a cysteine protease.

2. The modified serine protease proprotein of claim 1, wherein the serine protease is selected from porcine pancreatic elastase (PPE), human neutrophil elastase (ELANE), human cathepsin G (CTSG), and human proteinase 3 (PR3).

3. The modified serine protease proprotein of claim 1 or 2, which comprises, consists, or consists essentially of an amino acid sequence that is at least 80, 85, 90, 95, 98, or 100% identical to a sequence selected from Table S1, and which comprises or retains the heterologous protease cleavage site.

4. The modified serine protease proprotein of any one of claims 1-3, wherein the metalloprotease, aspartyl protease, or cysteine protease is selected from matrix metalloproteinase-12 (MMP12), cathepsin D (CTSD), cathepsin C (CTSD), and cathepsin L (CTSL).

5. The modified serine protease proprotein of claim 4, wherein the heterologous protease cleavage site is selected from Table S3.

6. The modified serine protease proprotein of claim 4, wherein the heterologous protease cleavage site is the MMP12 cleavage site of SEQ ID NO: 8.

7. The modified serine protease proprotein of claim 4, wherein the heterologous protease cleavage site is the CTSD cleavage site of SEQ ID NO: 11.

8. The modified serine protease proprotein of claim 4, wherein the heterologous protease cleavage site is the CTSC cleavage site of SEQ ID NO: 13.

9. The modified serine protease proprotein of claim 4, wherein the heterologous protease cleavage site is the CTSL cleavage site of SEQ ID NO: 14.

10. The modified serine protease proprotein of any one of claims 1-9, which does not substantially bind to a serine protease inhibitor (Serpin) in vitro or in vivo.

11. The modified serine protease proprotein of claim 10, wherein the Serpin includes alpha-1 antitrypsin (A1AT).

12. The modified serine protease proprotein of any one of claims 1-11, which is substantially inactive as a serine protease in its proprotein form.

13. The modified serine protease proprotein of any one of claims 1-12, wherein protease cleavage of the heterologous protease cleavage site, optionally at a cancer or tumor site in vivo, generates an active peptidase domain (or active serine protease domain), which has increased serine protease activity relative to the proprotein.

14. The modified serine protease proprotein of claim 13, wherein the serine protease activity of the active peptidase domain is increased by about or at least about 2-fold, 5-fold, 10-fold, 50-fold, 100-fold, 500-fold, or 1000-fold or more relative to that of the proprotein.

15. The modified serine protease proprotein of any one of claims 1-14, wherein protease cleavage of the heterologous protease cleavage site, optionally at a cancer or tumor site in vivo, generates an active peptidase domain (or active serine protease domain), which has increased cancer cell-killing activity relative to the proprotein.

16. The modified serine protease proprotein of claim 15, wherein the cancer cell-killing activity of the active peptidase domain is increased by about or at least about 2-fold, 5-fold, 10-fold, 50-fold, 100-fold, 500-fold, or 1000-fold or more relative to that of the proprotein.

17. The modified serine protease of any one of claims 5-16, wherein:
the serine protease is PPE, and the active peptidase domain comprises, consists, or consists essentially of an amino acid sequence that is at least 80, 85, 90, 95, 98, or 100% identical to residues 31-266 of SEQ ID NO: 1;
the serine protease is human ELANE, and the active peptidase domain comprises, consists, or consists essentially of an amino acid sequence that is at least 80, 85, 90, 95, 98, or 100% identical to residues 30-247 of SEQ ID NO: 2;
the serine protease is human CTSG, and the active peptidase domain comprises, consists, or consists essentially of an amino acid sequence that is at least 80, 85, 90, 95, 98, or 100% identical to residues 21-243 of SEQ ID NO: 3; or
the serine protease is human PR3, and the active peptidase domain comprises, consists, or consists essentially of an amino acid sequence that is at least 80, 85, 90, 95, 98, or 100% identical to residues 28-248 of SEQ ID NO: 4.

18. A modified porcine pancreatic elastase (PPE) proprotein, comprising in an N-terminal to C-terminal orientation, a signal peptide, a modified activation peptide relative to SEQ ID NO: 6 (wild-type PPE activation peptide), and a PPE peptidase domain, wherein the modified activation peptide is not substantially cleavable by trypsin and comprises a heterologous protease cleavage site that is cleavable by a protease selected from a metalloprotease, an aspartyl protease, and a cysteine protease.

19. The modified PPE proprotein of claim 18, wherein the protease is selected from matrix metalloproteinase-12 (MMP12), cathepsin D (CTSD), cathepsin C (CTSC), and cathepsin L (CTSL).

20. The modified PPE proprotein of claim 18 or 19, wherein the heterologous protease cleavage site comprises, consists, or consists essentially of an amino acid sequence selected from Table S3.

21. The modified PPE proprotein of claim 20, wherein:
the heterologous protease cleavage site is selected from SEQ ID NOs: 8-10, and is cleavable by MMP12;
the heterologous protease cleavage site is selected from SEQ ID NOs: 11-12, and is cleavable by CTSD;
the heterologous protease cleavage site is SEQ ID NO: 13, and is cleavable by CTSC; or
the heterologous protease cleavage site is selected from SEQ ID NOs: 14-16, and is cleavable by CTSL.

22. The modified PPE proprotein of any one of claims 18-21, wherein the signal peptide comprises the amino acid sequence set forth in SEQ ID NO: 5, or a variant thereof, and wherein the PPE peptidase domain comprises the amino acid sequence set forth in SEQ ID NO: 7, or an amino acid sequence that is at least 80, 85, 90, 95, 98, or 99% identical to SEQ ID NO: 7.

23. The modified PPE proprotein of any one of claims 18-22, comprising, consisting, or consisting essentially of an amino acid sequence that is at least 80, 85, 90, 95, 98, or 100% identical to a sequence selected from Table S4, and which retains the heterologous protease cleavage site.

24. The modified PPE proprotein of any one of claims 18-23, which does not substantially bind to a serine protease inhibitor (Serpin) in vitro or in vivo, optionally wherein the Serpin includes alpha-1 antitrypsin (A1AT).

25. The modified PPE proprotein of any one of claims 18-24, which is substantially inactive as a serine protease in its PPE proprotein form.

26. The modified PPE proprotein of any one of claims 18-25, wherein protease cleavage of the heterologous protease cleavage site, optionally at a cancer or tumor site in vivo, generates an active PPE peptidase domain (or active PPE protein), which has increased serine protease activity relative to the PPE proprotein.

27. The modified PPE proprotein of claim 26, wherein the serine protease activity of the active PPE protein is increased by about or at least about 2-fold, 5-fold, 10-fold, 50-fold, 100-fold, 500-fold, or 1000-fold or more relative to that of the PPE proprotein.

28. The modified PPE proprotein of any one of claims 18-27, wherein protease cleavage of the heterologous protease cleavage site, optionally at a cancer or tumor site in vivo, generates an active PPE peptidase domain (or active PPE protein), which has increased cancer cell-killing activity relative to the PPE proprotein.

29. The modified PPE proprotein of claim 28, wherein the cancer cell-killing activity of the active PPE protein is increased by about or at least about 2-fold, 5-fold, 10-fold, 50-fold, 100-fold, 500-fold, or 1000-fold or more relative to that of the PPE proprotein.

30. A recombinant nucleic acid molecule encoding the modified serine protease proprotein, optionally the modified PPE proprotein, of any one of claims 1-29, a vector comprising the recombinant nucleic acid molecule, or a host cell comprising the recombinant nucleic acid molecule or the vector.

31. A method of producing a modified serine protease proprotein, optionally a modified PPE proprotein, comprising culturing the host cell of claim 30 under culture conditions suitable for the expression of the proprotein, and isolating the proprotein from the culture.

32. A pharmaceutical composition, comprising the modified serine protease proprotein, optionally the modified PPE proprotein, of any one of claims 1-29, or an expressible polynucleotide encoding the proprotein, and a pharmaceutically acceptable carrier.

33. A method of treating, ameliorating the symptoms of, and/or reducing the progression of, a cancer in a subject in need thereof, comprising administering to the subject pharmaceutical composition of claim 32.

34. The method of claim 33, wherein the cancer is a primary cancer or a metastatic cancer, and is selected from one or more of melanoma (optionally metastatic melanoma), breast cancer (optionally triple-negative breast cancer, TNBC), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

35. The method of claim 33 or 34, wherein the modified serine protease proprotein, optionally the modified PPE proprotein, is activated by protease cleavage of the heterologous protease cleavage site in a cell or tissue, optionally a cancer or tumor cell or tissue, to generate an active peptidase domain, optionally an active PPE peptidase domain, wherein the active peptidase domain has increased serine protease activity and/or cancer cell-killing activity relative to the proprotein.

36. The method of claim 35, wherein the active peptidase domain increases cancer cell-killing in the subject by about or at least about 2-fold, 5-fold, 10-fold, 50-fold, 100-fold, 500-fold, or 1000-fold or more relative to a control or reference.

37. The method of claim 35 or 36, wherein the active peptidase domain results in tumor regression in the subject, optionally as indicated by a statistically significant decrease in the amount of viable tumor or tumor mass, optionally at least about a 10%, 20%, 30%, 40%, 50% or more decrease in tumor mass.

38. The method of any one of claims 33-37, comprising administering the pharmaceutical composition to the subject by parenteral administration.

39. The method of claim 38, wherein the parenteral administration is intravenous administration.
